(19) United States
(12) Patent Application Publication
(10) Pub. No.: US 2005/0055012 A1
(43) Pub. Date: Mar. 10, 2005

(54) Convertible multi-lumen catheter

(76) Inventor: Scott O. Trerotola, Newton Square, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/658,647

(22) Filed: Sep. 9, 2003

(51) Int. Cl. A61M 31/00

(52) U.S. Cl. 604/508; 604/43

(57) ABSTRACT

A convertible multi-lumen catheter that may be used for hemodialysis or other indications involving infusion and/or withdrawal of fluids from the body. Unlike existing catheters having a set number of lumens which may limit their utility as both short and long-term venous vascular devices, the catheter of the invention allows one or more additional lumens during the acute phase of catheter use with removal of these lumens (i.e. conversion) for more permanent use. A typical example of this would be a triple lumen device for hemodialysis and antibiotic therapy during an acute infection with conversion to a chronic dual lumen hemodialysis catheter after successful treatment of the infection. The lumen is permanently or semi-permanently blocked using a biocompatible plastic obturator that is inserted into the unused lumen and locked and/or glued into place.

### FIELD OF THE INVENTION

The invention described herein relates to a multi-lumen catheter that has an obturator for permanently or semi-permanently blocking an unused lumen while treatment continues in another lumen, thereby minimizing risk of infection and eliminating the need to remove the catheter until all treatments are completed.

### BACKGROUND OF THE INVENTION

Over 250,000 patients undergo hemodialysis in the United States each year, with many more worldwide. Millions of venous access devices are also placed annually for indications which include cancer therapy, chronic parenteral nutrition, and the like. Presently, hemodialysis and infusion needs are met by placement of a variety of venous access devices including PICC lines, ports, and tunneled catheters that are generally but not always tunneled subcutaneously. Presently, a patient in need of such a device receives a catheter consisting of one or more lumens where the catheter configuration is chosen based on the patient's needs at the time of catheter placement. For example, a patient needing infusion of two incompatible medications will need at least two lumens. Patients undergoing hemodialysis need at least two lumens for hemodialysis but may need additional lumens for medications as the hemodialysis lumens are dedicated for that purpose. One problem with this approach is that patients' needs may vary from the time of catheter placement to a later time. Typically, patients need more lumens during an acute phase of their illness and fewer lumens during a later phase. For example, a patient with cancer may need multiple incompatible drugs for induction chemotherapy and therefore multiple lumens, but later needs a fewer number of lumens for chronic maintenance therapy.

In addition to these considerations, it is highly desirable to keep the number of lumens in a catheter to the minimum number required for sufficient therapy. The reason for this is that as the number of lumens increases, the risk of infection increases as well. Infection is one of the most feared complications of venous access devices and accounts for significant morbidity and mortality in patients receiving such devices. Thus, there is a need for a device which would allow for a reduction in the number of lumens once the additional lumens were no longer needed with an attendant decrease in the risk of infection long-term.

The prior art is replete with multi-lumen catheters (e.g., U.S. Pat. Nos. 5,221,256; 6,001,079; 5,807,311; 4,995,865; 4,808,155; 4,643,711; 4,543.087; 5,378,230), but none of these catheters permits the user to reduce the number of lumens without changing the device. Mahurkar and others have described multiple devices for hemodialysis with various luminal configurations, all of which are fixed and not convertible to fewer lumens. Mahurkar's patents and other patents concerning hemodialysis catheters concentrate almost exclusively on the need to maintain satisfactory pressures and flows for hemodialysis, ignoring the need for additional infusion therapy which ideally would be provided by the same catheter. Certainly, triple, quadruple and higher number multi-lumen catheters have been described for varying purposes, including infusion, monitoring, an the like. Again, while the various prior art patents describe the physical attributes of the catheters and lumens, none describes the ability to change the number of lumens as patient's needs change. Several patents have described devices for increasing the number of lumens (U.S. Pat. Nos. 6,013,068; 5,149,330), but this is fundamentally different from reducing the number of lumens in both intent and practice.

Catheters with collapsible guide wire lumens are also known in the art (e.g., U.S. Pat. No. 6,450,987; US 2003/0023229). For example, Maginot discloses in U.S. Pat. No. 6,156,016 and US 2002/0091362 the use of a guide catheter that permits replacement of an inner guide catheter when it becomes dysfunctional. However, none of these systems reduces the risk of infection by blocking off the unused lumen when it is not in use. A multi-lumen catheter is thus desired that permits one or more lumens to be used for acute treatments and selectively blocked when not needed, while allowing use of the other lumens to continue. The present invention has been designed to address this pressing need in the art.

### SUMMARY OF THE INVENTION

A convertible multi-lumen catheter that addresses the above-referenced needs in the art comprises an elongated tube having at least two lumens and a removable obturator configured to block a lumen when it is not in use, thereby minimizing risk of infection. The obturator may be removable so that its lumen may be used to inject fluids and the like as needed in more acute settings. The obturator is preferably made of a biocompatible plastic of sufficient stiffness for insertion into a lumen and may also have a locking mechanism, such as a luer lock, on a distal end that removably connects the obturator to the catheter. The locking mechanism is configured such that the distal end of the obturator is flush with a distal end of catheter when the locking mechanism is engaged. The obturator may extend the length of the lumen or may extend partially into the lumen. In the latter case, the obturator may have a mark thereon at a predetermined point so as to identify an amount of dead space in the lumen remaining when the obturator is fully inserted into the lumen. If it is desired to permanently block the lumen, a biocompatible adhesive may be injected into the dead space of the lumen prior to insertion of the obturator, thereby bonding the obturator to the catheter.

A convertible multi-lumen catheter assembly in accordance with the invention may also include a hub attached to a distal end of the catheter. The hub may include a marking identifying the amount of dead space in the lumen and a fitting that communicates with the lumen that is configured to match a syringe containing an amount of the adhesive sufficient to fill the amount of dead space in the lumen.

In a first configuration, the convertible multi-lumen catheter of the invention has at least two lumens, a first lumen for inserting fluids into a patient and a second lumen for inserting fluids into a patient. Either lumen or both may be adapted to accept the obturator.

In a second configuration, the convertible multi-lumen catheter of the invention has at least three lumens, a first lumen adapted to insert antibiotics into a patient or for accepting a guide wire, a second lumen for inserting fluids into the patient, and a third lumen for removing fluids from the patient. The obturator may be accepted in any one or all of the lumens to selectively block the lumens as desired.

In a third configuration, the lumen into which the obturator is inserted is adapted to collapse when not in use and the obturator is not inserted.

The invention also encompasses a method of treating a patient with a catheter having at least two lumens. Such a method in accordance with the invention includes the steps of inserting the catheter into the patient, applying a first treatment to the patient via a first lumen of the catheter, applying a second treatment to the patient via a second lumen of the catheter, and sealing one of the lumens when it is not in use while treatment continues in the other lumen. The sealing may be performed by inserting an obturator into the lumen when it is not in use.

Such a method in accordance with the invention also includes the further step of removably locking a distal end of the obturator in the lumen using a locking mechanism. Preferably, the locking mechanism is configured such that the distal end of the obturator is flush with a distal end of the catheter when the locking mechanism is engaged. In one embodiment of the invention, the obturator leaves a dead space in the lumen when the obturator is fully inserted. This method may include the further step of inserting a biocompatible adhesive into the dead space of the lumen prior to insertion of the obturator in the inserting step. In this embodiment, the adhesive permanently bonds the obturator to the catheter to prevent further usage of the blocked lumen and any further risk of infection.

The method of permanently blocking a lumen in accordance with the invention may also include the further step of attaching a hub to a distal end of the catheter, where the hub identifies the amount of dead space in the lumen and has a fitting that communicates with the lumen. The fitting is preferably configured to match a syringe containing an amount of the adhesive sufficient to fill the amount of dead space in the lumen.

The method of the invention may be used for catheters with two or more lumens. In the two lumen configuration, the first treatment applying step may comprise the step of inserting fluids into the patient via the first lumen and the second treatment applying step may comprise the step of inserting fluids into the patient via the second lumen. In the case of three or more lumens, on the other hand, the first treatment applying step may comprise the step of inserting antibiotics or a guide wire into the first lumen for insertion into the patient and the second treatment applying step may comprise the step of inserting fluids into the patient via the second lumen. In the latter case, the obturator inserting step includes the step of inserting the obturator into the first lumen after cessation of the application of the antibiotics or the removal of the guide wire from the first lumen.

### BRIEF DESCRIPTION OF THE DRAWINGS

The above-mentioned features and advantages of the invention will be apparent from the following detailed description in conjunction with the drawings, of which:

FIGS. 1(A) and 1(B) illustrate a cross-section of a three lumen catheter including a guide wire in the third lumen (FIG. 1(A)) and the same three lumen catheter with the guide wire removed from the third lumen leaving a dead space (FIG. 1(B)).

FIGS. 2(A) and 2(B) illustrate a cross-section of a four lumen catheter including guide wires in the third and fourth lumens (FIG. 2(A)) and the same four lumen catheter with the guide wires removed from the third and fourth lumens leaving dead spaces (FIG. 2(B)).

FIGS. 3(A) and 3(B) illustrate a cross-section of a catheter including a center lumen for accepting a guide wire (FIG. 3(A)) and the same catheter with the guide wire removed so that the common adjacent walls of the remaining lumens return to their initial position, thereby closing off the space that held the guide wire (FIG. 3(B)).

FIGS. 4(A) and 4(B) illustrate the proximal end of a multi-lumen catheter in accordance with an embodiment of the invention with the obturator partially inserted (FIG. 4(A)) and fully inserted (FIG. 4(B)).

FIGS. 5(A) and 5(B) illustrate the distal end of a multi-lumen catheter in accordance with an embodiment of the invention with the obturator inserted (FIG. 5(A)) and removed (FIG. 5(B)).

FIGS. 6(A) through 6(C) illustrate the proximal end of the multi-lumen catheter in accordance with an embodiment of the invention where the obturator extends the length of the lumen, where FIG. 6(A) illustrates the proximal end of the locked obturator, FIG. 6(B) illustrates a cross-section of the catheter of FIG. 6(A), and FIG. 6(C) illustrates the extension of the obturator through the lumen.

FIG. 6(D) illustrates the obturator of the embodiment of FIGS. 6(A) through 6(C).

FIGS. 7(A) and 7(B) illustrate the proximal end of the multi-lumen catheter in accordance with an embodiment of the invention where the obturator extends partially into the lumen so as to leave a dead space, where FIG. 7(A) illustrates the catheter prior to insertion of the obturator and FIG. 7(B) illustrates the catheter with the obturator fully inserted and glued into place.

FIG. 8 illustrates the proximal end of the multi-lumen catheter in accordance with an embodiment of the invention where the hub of the lumen for accepting the obturator has a non-luer fitting such as (a) or (b) designed to mate with a matching syringe or other delivery system containing biocompatible adhesive for injection into the dead space of the lumen.

### DETAILED DESCRIPTION OF ILLUSTRATIVE EMBODIMENTS

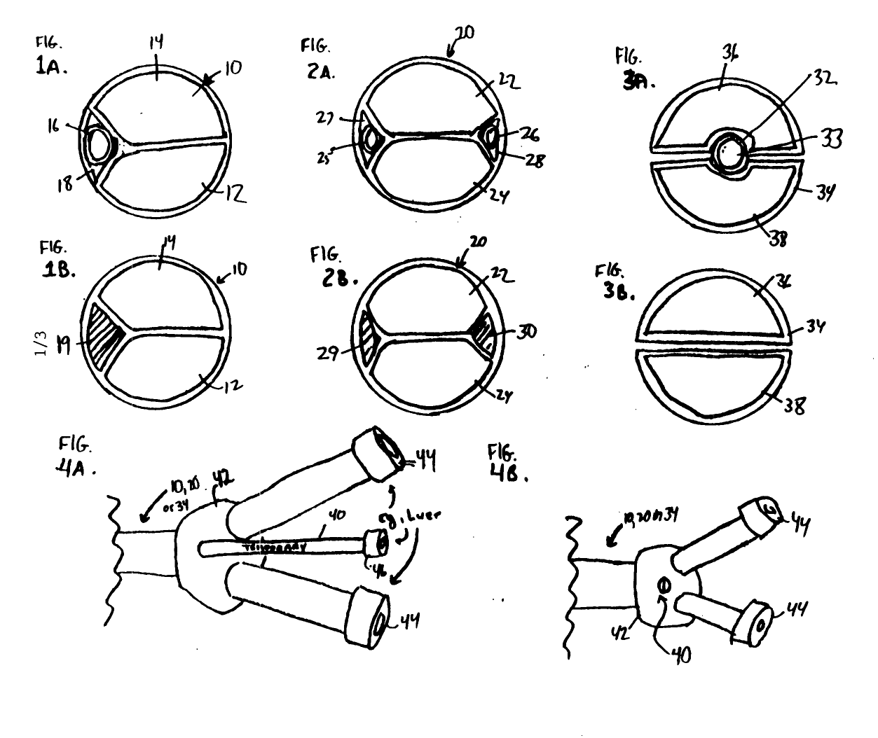

A detailed description of exemplary embodiments of the present invention will now be described with reference to FIGS. 1-8. Although this description provides detailed examples of possible implementations of the present invention, it should be noted that these details are intended to be exemplary and in no way delimit the scope of the invention.

Several possible embodiments of a convertible multi-lumen catheter in accordance with the invention are shown in the figures. For example, FIGS. 1(A) and 1(B) illustrate a cross-section of a three lumen catheter 10 including two fixed lumens 12, 14 and a guide wire 16 in a third lumen 18 (FIG. 1(A)). As shown in FIG. 1(B), dead space 19 remains in the catheter 10 when the guide wire is removed from the third lumen. Similarly, FIGS. 2(A) and 2(B) illustrate a cross-section of a four lumen catheter 20 including two fixed lumens 22, 24 with guide wires 25, 26 in the third and fourth lumens 27, 28, respectively. As shown in FIG. 2(B), dead spaces 29, 30 remain in the catheter 20 when the guide wires are removed from the third and fourth lumens. Thus, in each of these embodiments, dead space remains when the guide wire is not inserted or the lumen is not otherwise used for fluid insertion.

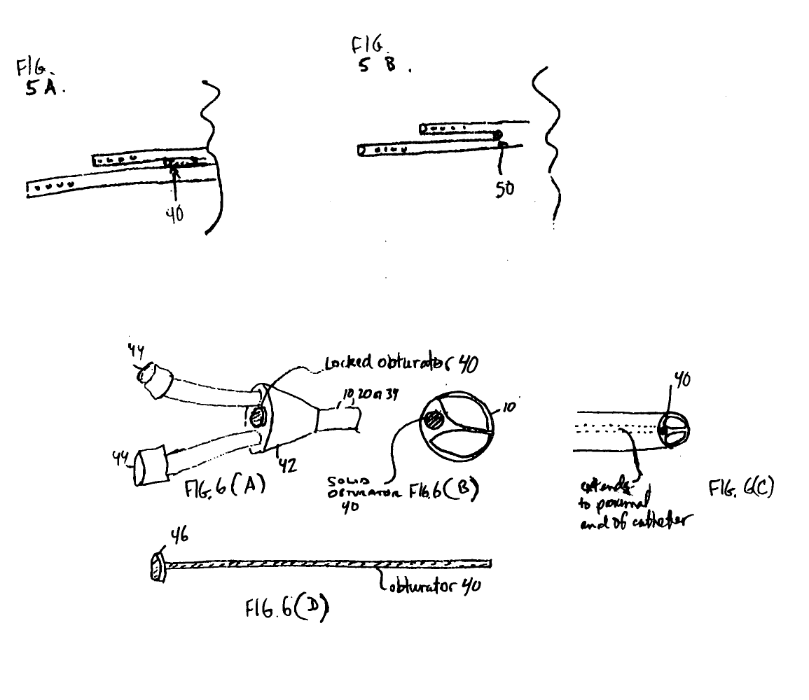

Alternatively, FIGS. 3(A) and 3(B) illustrate a cross-section of a catheter 34 including a center lumen 32 for accepting a guide wire 33 (FIG. 3(A)). As shown in FIG. 3(B), when the guide wire 33 is removed from the catheter 34, the lumen 32 collapses and the remaining lumens 36, 38 conform to fill the dead space. This embodiment might be desirable for maintaining the highest possible flow rates through the remaining lumens 36, 38, such as is desirable in hemodialysis and apheresis.

Those skilled in the art will appreciate that a significant consideration is how to maintain hemostasis and aerostasis once the additional lumens have been removed. This may be accomplished by means of hemostasis valves such as are found in vascular sheaths or more permanently through the use of adhesives such as silicone or other medical grade adhesive. For example, FIGS. 4(A) and 4(B) illustrate the proximal end of a multi-lumen catheter in accordance with a first embodiment of the invention with the obturator 40 partially inserted (FIG. 4(A)) through a luer hub assembly 42 into the lumen and fully inserted into the lumen (FIG. 4(B)). As illustrated, luer connections 44 may be included for communication with each lumen. As desired, the obturator 40 may also have a luer lock mechanism 46 for locking the obturator to the catheter via the luer hub assembly 42.

Tip configuration for the catheters 10, 20, or 34 would depend on the medical use anticipated. This may include step and/or split tip multi side hole configurations for hemodialysis, or more conventional trimmable tips as used in oncology catheters. One possible configuration is shown in FIG. 5, with an obturator in place in a third lumen (FIG. 5A) and after removal (FIG. 5B) with closed slit valve 50.

There are several possible mechanisms for permanently or semi-permanently blocking one or more unused lumens using the techniques of the invention. Those skilled in the art will appreciate that although a single removable lumen is shown in the figures that any number of removable lumens may be implemented using the techniques of the invention. Moreover, those skilled in the art will appreciate that the invention may be used with any type of conventional multi-lumen catheter, not just those illustrated.

For example, FIGS. 6(A) through 6(C) illustrate the proximal end of the multi-lumen catheter of FIG. 4 where the obturator 40 extends the length of the lumen and is locked at the proximal end of the catheter assembly (FIG. 6(A)). FIG. 6(B) illustrates a cross-section of the catheter of FIG. 6(A) with the solid obturator 40 blocking the unused lumen. FIG. 6(C) illustrates the extension of the obturator 40 through the lumen, while FIG. 6(D) illustrates the obturator 40. Preferably, obturator 40 is made of a biocompatible plastic of sufficient stiffness to easily pass into the catheter as shown. The locking mechanism 46 may be a luer lock, which connects the obturator 40 to the catheter hub 42, filling the unused lumen as shown in FIG. 6(B) and rendering the tip portion flush as shown in FIG. 6(C). Those skilled in the art will appreciate that it may be desirable for the locking mechanism to not be a luer lock to prevent unintended use of the lumen after the catheter has been removed from the patient. Many locking mechanisms are known in the art that would be suitable for this purpose. However, having a luer hub integrated into the catheter hub 42 also might be desirable as an alternate embodiment.

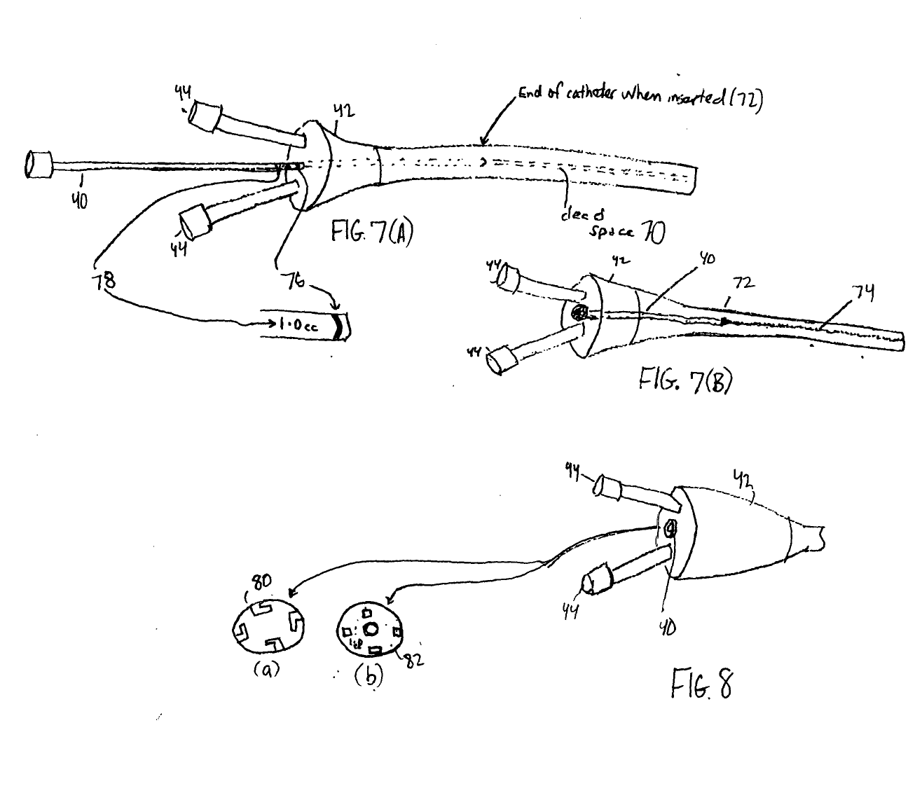

FIGS. 7(A) and 7(B) illustrate the proximal end of the multi-lumen catheter in accordance with an embodiment of the invention where the obturator 40 extends partially into the lumen so as to leave a dead space 70 beyond the point 72 where the obturator 40 ends when fully inserted. FIG. 7(A) illustrates the catheter prior to insertion of the obturator 40, and FIG. 7(B) illustrates the catheter with the obturator 40 fully inserted and glued into place by adhesive 74. In the embodiment of FIG. 7, a combination of obturator 40 and biocompatible adhesive 74 are preferred if it is desired to permanently block the lumen. The adhesive 74 could be delivered using a blunt cannula and syringe similar to that found in commercially available Hickman catheter repair kits or by other means. The combination of hemostasis valves and medical grade adhesive also may be desirable, but it is not necessarily desirable or required to completely fill the lumen with adhesive 74 as a blood clot would rapidly form in and occlude any remaining dead space.

As shown in FIG. 7(A), the obturator 40 may have markings on it at a predetermined point 76 with the remaining dead space of the lumen indicated at 78. An appropriate adhesive in a Luer tip syringe could be connected to the removable obturator 40 and the designated dead space volume injected after priming the catheter (FIG. 7(A)) to fill its dead space. The obturator 40 is then inserted and locked into place as described above. However, in this embodiment the obturator 40 is permanently bonded to the convertible catheter because of the adhesive 74. This double occlusion (adhesive 74 plus obturator 40) is an extra safety check against accidental dislodgement of the obturator 40, which could result in blood loss or air embolus.

FIG. 8 illustrates the proximal end of the multi-lumen catheter in accordance with an embodiment of the invention where the hub 42 of the lumen for accepting the obturator 40 has a non-luer fitting such as fitting 80(a) or fitting 82(b) designed to mate with a matching syringe or other delivery system containing biocompatible adhesive for injection into the dead space of the lumen. By so avoiding luer fittings for injecting the adhesive 74, accidental occlusion of an undesired lumen may be prevented. As in the embodiment of FIG. 7, the dead space volume may be written on the fitting of the hub 42 and a matching syringe or other delivery system attached to the fitting whereby the appropriate volume of biocompatible adhesive is injected to fill and permanently occlude the lumen. A short obturator or cap may be attached to the fitting 80, 82 to cover the lumen once the adhesive is injected.

From the above description, it should be readily apparent that numerous other modifications and combinations of the above disclosure may be made without departing from the scope of the present invention. For example, the obturator may be threaded at its proximal end so that it may be screwed into place. The techniques of the invention may be used with catheters having any number of lumens and any tip and hub arrangement. Further, the methods described herein are intended as specific implementations only and are not intended to delimit the scope of the invention, which should instead be understood with reference to the following claims.

1. A multi-lumen catheter comprising an elongated tube having at least two lumens and a removable obturator configured to block a first lumen when said first lumen is not in use.

2. A catheter as in claim 1 wherein said obturator is made of a biocompatible plastic of sufficient stiffness for insertion into said first lumen.

3. A catheter as in claim 1 wherein said obturator comprises a locking mechanism on a distal end that removably connects said obturator to said tube and is configured such that said distal end of said obturator is flush with a distal end of said tube when said locking mechanism is engaged.

4. A catheter as in claim 3, wherein said locking mechanism is a luer lock.

5. A catheter as in claim 1, wherein said obturator has a mark thereon at a predetermined point so as to identify an amount of dead space in said first lumen when said obturator is fully inserted into said first lumen.

6. A catheter as in claim 5, further comprising a biocompatible adhesive that is injected into the dead space of said first lumen prior to insertion of said obturator, said adhesive bonding said obturator to said tube.

7. A catheter as in claim 6, further comprising a hub attached to a distal end of said tube, said hub identifying the amount of dead space in said first lumen, said hub having a fitting that communicates with said first lumen, said fitting configured to match a syringe containing an amount of said adhesive sufficient to fill the amount of dead space in said first lumen.

8. A catheter as in claim 1 having two lumens, said first lumen for inserting fluids into a patient and a second lumen for inserting fluids into a patient, said first lumen adapted to accept said removable obturator.

9. A catheter as in claim 1 having three lumens, said first lumen adapted to insert antibiotics into a patient or for accepting a guide wire, said first lumen adapted to accept said removable obturator, a second lumen for inserting fluids into a patient, and a third lumen for removing fluids from a patient.

10. A catheter as in claim 1 wherein said first lumen is adapted to collapse when not in use and said obturator is not inserted.

11. A method of treating a patient with a catheter having at least two lumens, comprising the steps of:
inserting the catheter into the patient;
applying a first treatment to the patient via a first lumen of the catheter;
applying a second treatment to the patient via a second lumen of the catheter; and
inserting a removable obturator into said one of said first and second lumens when said one lumen is not in use while treatment continues in the other of said first and second lumens, said obturator configured to block said one lumen when said one lumen is not in use.

12. A method as in claim 11 comprising the further step of removably locking a distal end of said obturator in said one lumen using a locking mechanism, wherein said locking mechanism is configured such that said distal end of said obturator is flush with a distal end of said catheter when said locking mechanism is engaged.

13. A method as in claim 11, wherein said obturator leaves a dead space in said one lumen when said obturator is fully inserted, comprising the further step of inserting a biocompatible adhesive into said dead space of said one lumen prior to insertion of said obturator in said inserting step, said adhesive bonding said obturator to said catheter.

14. A method as in claim 13, further comprising the step of attaching a hub to a distal end of said catheter, said hub identifying the amount of dead space in said one lumen and having a fitting that communicates with said one lumen, said fitting configured to match a syringe containing an amount of said adhesive sufficient to fill the amount of dead space in said one lumen.

15. A method as in claim 11, wherein said first treatment applying step comprises the step of inserting fluids into the patient via said first lumen and said second treatment applying step comprises the step of inserting fluids into the patient via said second lumen.

16. A method as in claim 11, wherein said first treatment applying step comprises the step of inserting antibiotics or a guide wire into the first lumen for insertion into the patient and said second treatment applying step comprises the step of inserting fluids into the patient via said second lumen.

17. A method as in claim 16, wherein said obturator inserting step includes the step of inserting said obturator into said first lumen after cessation of the application of said antibiotics or the removal of said guide wire.

18. A method of treating a patient with a catheter having at least two lumens, comprising the steps of:
inserting the catheter into the patient;
applying a first treatment to the patient via a first lumen of the catheter;
applying a second treatment to the patient via a second lumen of the catheter; and
sealing one of said first and second lumens when said one lumen is not in use while treatment continues in the other of said first and second lumens.

19. A method as in claim 18 wherein said sealing step comprises the steps of inserting a removable obturator into said one lumen and locking a distal end of said obturator in said one lumen using a locking mechanism, wherein said locking mechanism is configured such that said distal end of said obturator is flush with a distal end of said catheter when said locking mechanism is engaged.
